(19) United States
(12) Patent Application Publication
(10) Pub. No.: US 2006/0052846 A1
(43) Pub. Date: Mar. 9, 2006

(54) Physical electrotherapy device

(76) Inventors: Chieh-Lin Liu, Taichung City (TW); Hao-Hua Hung, Taichung City (TW)

Correspondence Address: RABIN & BERDO, P.C., Suite 500, 1101 14th Street, N.W., Washington, DC, 20005, US

(21) Appl. No.: 10/935,216

(22) Filed: Sep. 8, 2004

(51) Int. Cl. A61N 1/18

(52) U.S. Cl. 607/72

(57) ABSTRACT

A physical electrotherapy device comprised of a pulse switch selector; a first section pure pulse wave generating circuit; an intermittent pulse wave generating circuit; a third section high/low frequency alternating wave generating circuit; a rectifier, wave filter, and voltage stabilizer circuit; an LED output indication circuit; and a voltage regulating and efficiency enhancing output circuit. As such, the resulting electrotherapy device is non-injurious and capable of human body strengthening and health care maintenance.

### BACKGROUND OF THE INVENTION

1) Field of the Invention

The invention herein relates to a physical electrotherapy device that provides for appropriate human physiological therapeutic applications such as stimulating sole reflex areas, improving blood circulation, fatigue recovery, and muscles and tendons treatment. Utilized to strengthen bodily resistance as well as restore and increase the natural healing capabilities originally endowed the human body, the physical electrotherapy device herein can be used for both medical treatment and health maintenance.

2) Description of the Prior Art

Of the electrotherapeutic models observable at present, most are outdated legacy approaches, often low frequency electrotherapeutic methods that are only capable of electrically stimulating shallow regions of the human body, with such conventional low frequency machines typically having the following shortcomings:
1. Electric impact stimulation: Overly intense, tingling sensation, and only in shallow flesh regions.
2. Wave form rigidity: Inflexible and cannot be adjusted to accommodate the greater or lesser resistance of each person.
3. Acute wave form: Shallow level, slow pulse wave speed, cannot sense skin pain or nerve irritation, and stinging sensation.
4. High saturation: No anti-weighting space, harsh.
5. Electrical discharge slow: Absorption too gradual, slow to reach afflicted area, sluggish pulse wave speed results in excessive nerve pain and unbearable stinging.
6. Increased pressure multiple too high; the higher the voltage, the less suitable for the human body.

Therefore, the applicant of the invention herein, deeply aware of the shortcomings and inadequacies of the prior art and based on many years of experience gained while engaged in the relevant field, conducted continuous research and development as well as refinement, culminating in the successful development of the present invention which is submitted as a new patent application.

### SUMMARY OF THE INVENTION

The primary objective of the invention herein is to provide a physical therapy device, the overall design of which in addition to the effective treatment of sole reflex areas is also capable of the deep stimulation of acupuncture channels in the muscles and tendons of the human body and, therefore, capable of reaching the muscle and tendon blood vessels and, furthermore, at the same time stimulating electro-therapy occurs, allows the production of regular spasms in the lower limbs of persons that in conjunction with pulleys at the rear extent of supporting footwear causes the cyclic movement of the lower limbs without creating interference to achieve the natural extension of the knee joint, thereby preventing joint degeneration; as such, the present invention has comprehensive medical treatment efficacy.

To enable the examination committee a further understanding of the structural features, operation, and other items of the present invention, the brief description of the drawings below are followed by the detailed description of the invention herein.

### BRIEF DESCRIPTION OF THE DRAWINGS

FIG. 1 is a schematic drawing.

FIG. 2 is a block diagram.

FIG. 2-A is a partial, magnified drawing of the pulse switch selector and the generating oscillator circuit.

FIG. 2-B is a partial, magnified drawing of the oscillator circuit.

FIG. 2-C is a partial, magnified drawing of the rectifier, wave filter, and voltage stabilizer circuit.

FIG. 2-D is a partial, magnified drawing of the LED output indication circuit; and the voltage output circuit.

FIG. 2-E is a partial, magnified drawing of the reverse logic gates.

FIG. 3 is drawing of the first section pure pulse wave.

FIG. 4 is a drawing of the second section intermittent wave.

FIG. 5 is a drawing of the third section high/low frequency alternating wave.

### DETAILED DESCRIPTION OF THE INVENTION

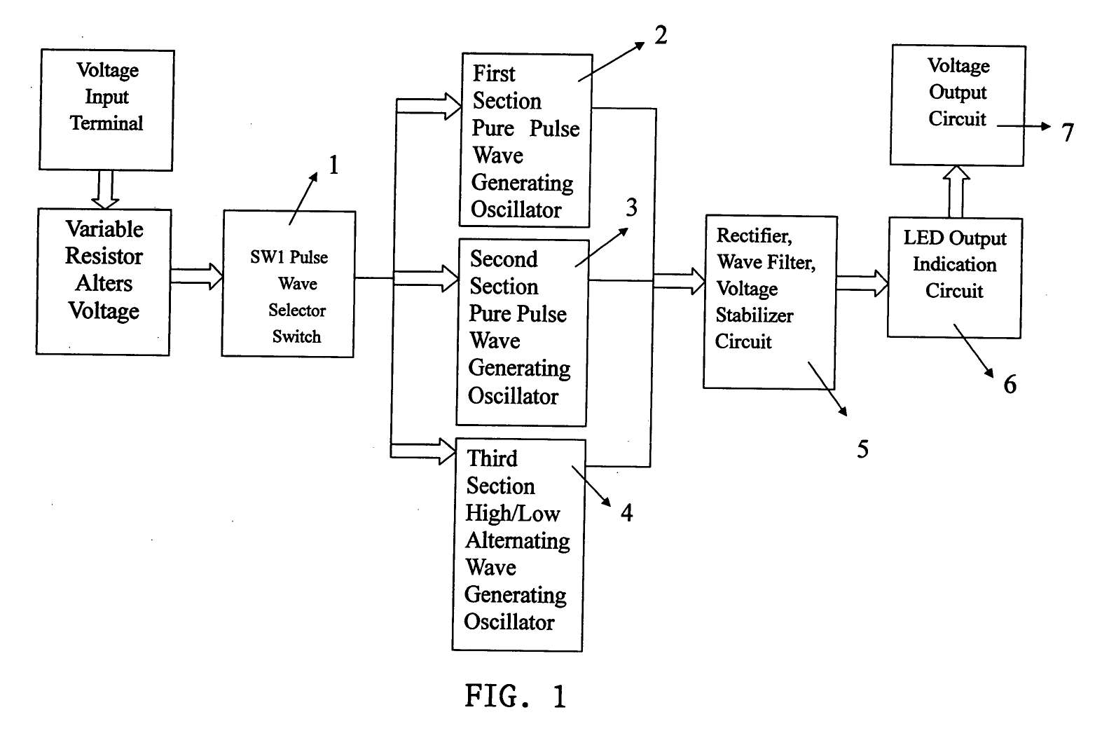

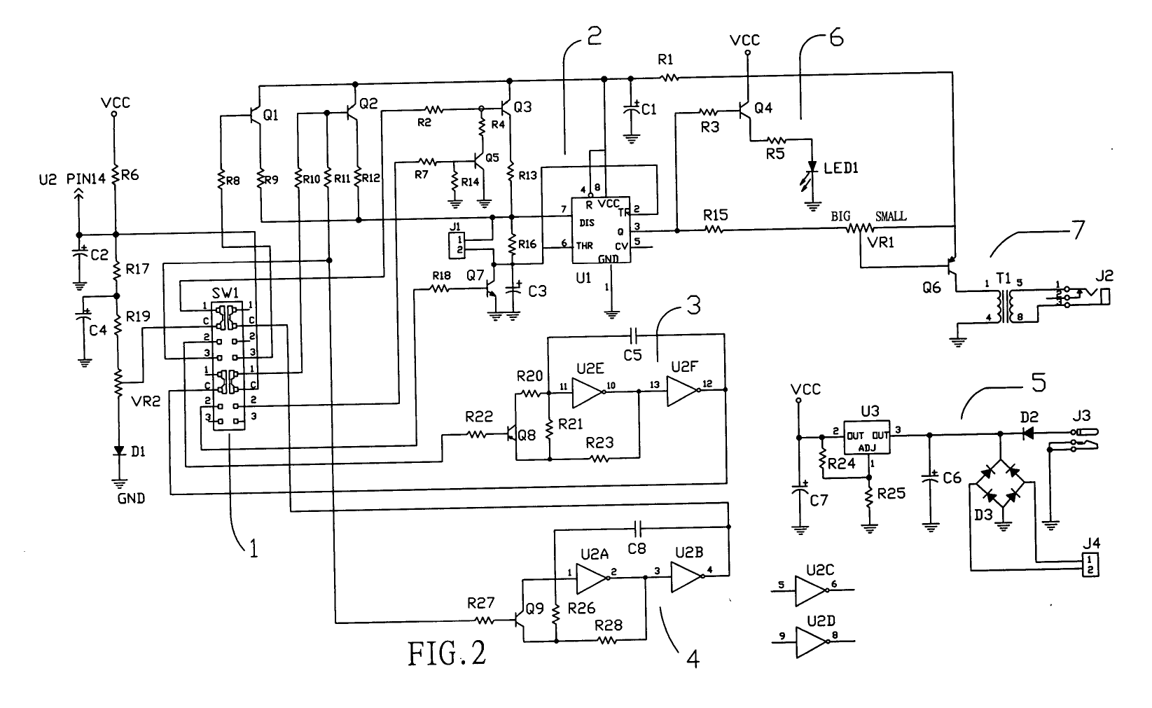

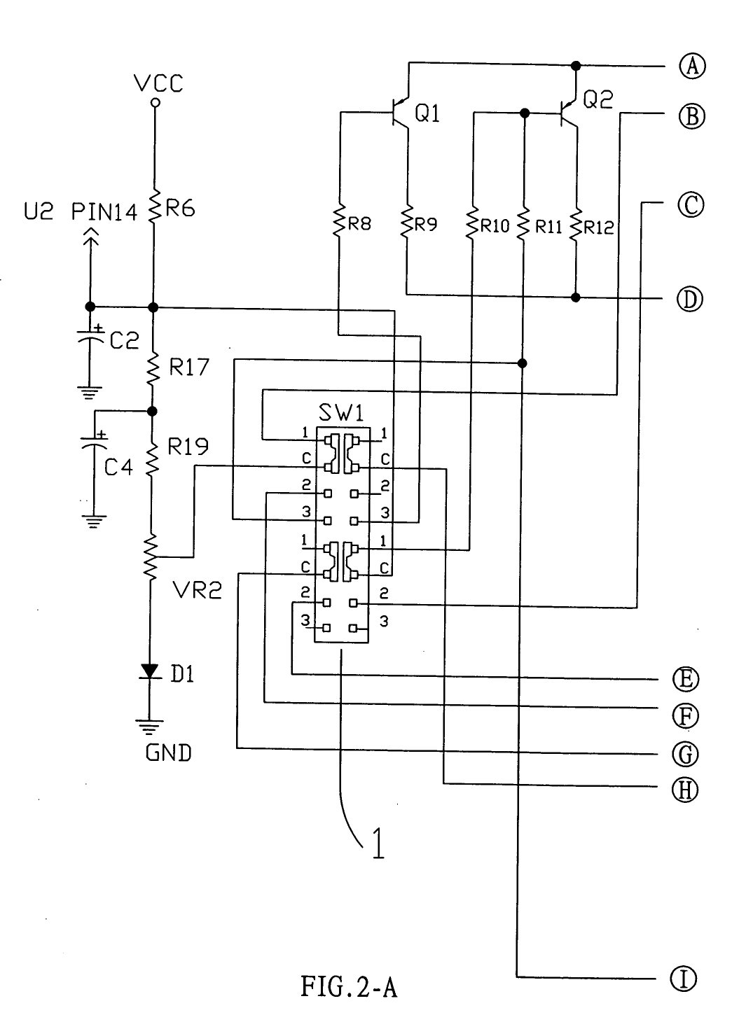

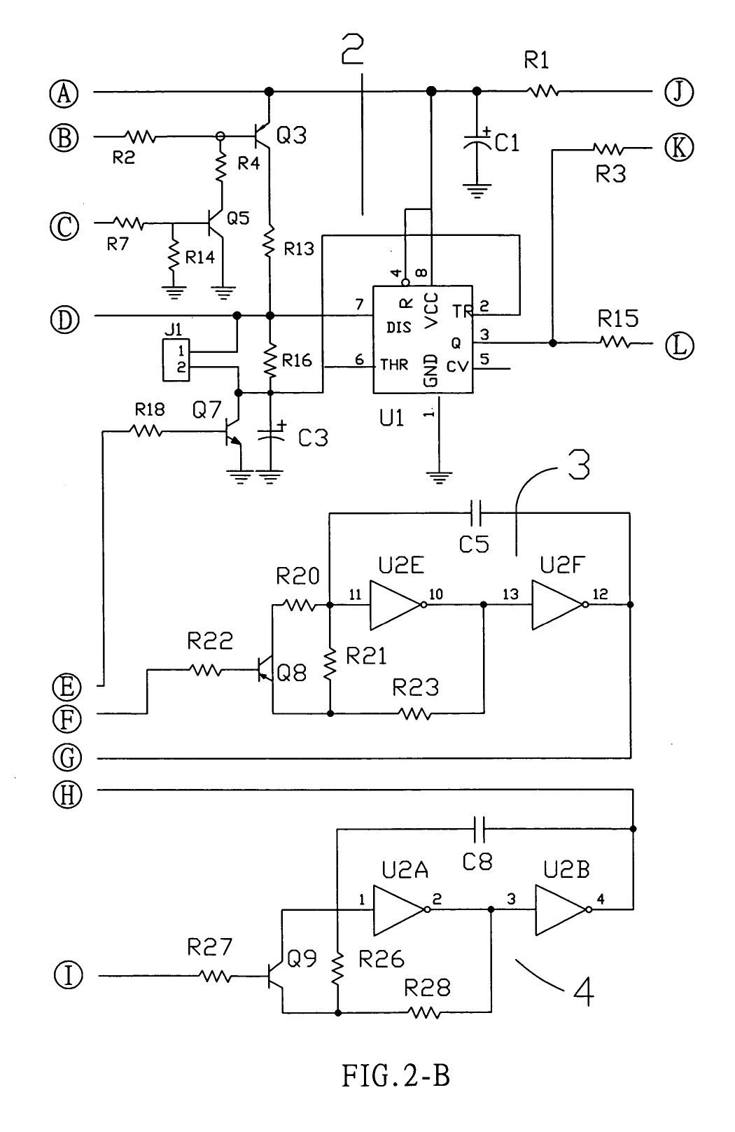

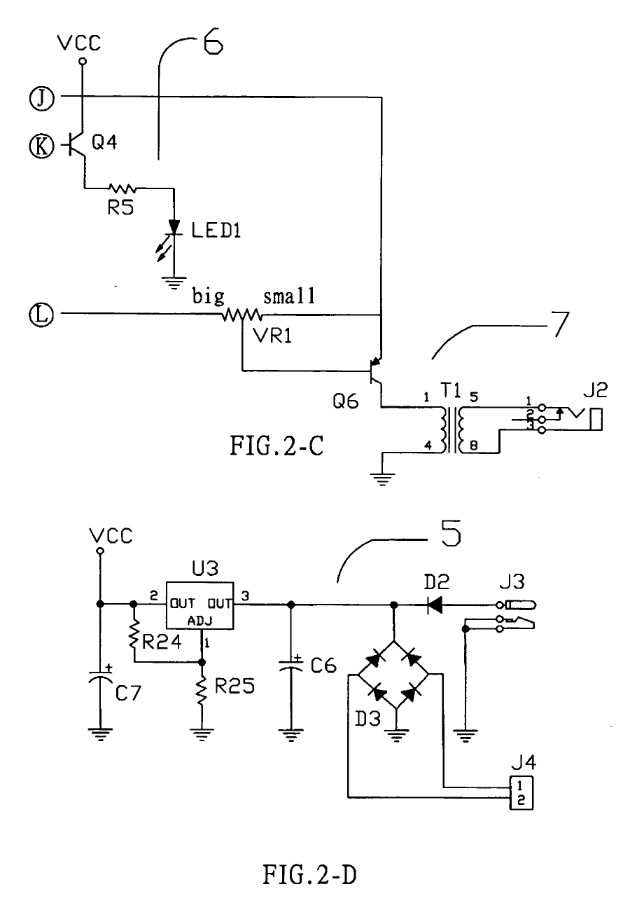

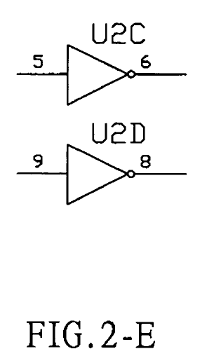

Refer to FIG. 1, the schematic drawing of the invention herein. FIG. 2 is a block diagram of the invention herein. FIG. 2-A is a partial, magnified drawing of the pulse switch selector and the generating oscillator circuit. FIG. 2-B is a partial, magnified drawing of the oscillator circuit. FIG. 2-C is a partial, magnified drawing of the rectifier, wave filter, and voltage stabilizer circuit. FIG. 2-D is a partial, magnified drawing of the LED output indication circuit; and the voltage output circuit. FIG. 2-E is a partial, magnified drawing of the reverse logic gates. The present invention is comprised of a pulse switch selector 1; a first section pure pulse wave 2; a second section intermittent pulse wave 3; a third section high/low frequency alternating wave 4; a rectifier, wave filter, and voltage stabilizer circuit 5; an LED output indication circuit 6, and a voltage output circuit 7, wherein:

The pulse switch selector 1 (SW1) is utilized to selectively toggle between three different outputted wave forms.

The first section pure pulse wave 2 (an oscillator) consists of an integrated circuit U1, U1 internally including reverse logic gates U2C and U2D, resistors R13 and R16, and a capacitor C3 that enable the generation of a fundamental pulse wave, the lowest frequency of which is 1.2 Hz; this section toggled by the pulse switch selector (SW1).

The second section is an intermittent pulse wave generating oscillator 3 consisting of a square wave oscillator circuit, an intermittent wave outputting fundamental frequency circuit, and a pulse wave frequency control circuit, wherein the square wave oscillator circuit structure consists of reverse logic gates U2E and U2F, a capacitor C5, and resistors R21 and R22; when the second section intermittent wave has output, the fundamental wave frequency consists of control by a transistor Q5 and resistors R4, R7, and R14; the pulse wave frequency circuit structure consists of a controlling transistor Q8 and resistors R20 and R22, and the switch circuit structure consists of a controlling transistor Q7 and a resistor R18, which are utilized to control the second section intermittent wave form, this section utilizing the pulse switch selector (SW1) for toggling, the fundamental frequency of which is approximately 70 Hz and the slowest period is one alternation every two seconds.

The third section is a high/low frequency alternating wave generating oscillator 4 consisting of a square wave oscillator circuit, a high/low frequency control circuit, and a pulse wave frequency control circuit, wherein the square wave oscillator circuit structure consists of reverse logic gates U2A and U2B, a capacitor C8, and resistors R26 and R28; the high/low frequency control circuit consists of transistors Q1 and Q2 and resistors R8, R9, R11, and R12 that shape the third section high/low frequency alternating waves, and the pulse wave frequency circuit consists of a controlling transistor Q9 and resistor R27, this section utilizing the pulse switch selector (SW1) for toggling, the slowest duration, low frequency of which is approximately 1 Hz, the highest frequency is approximately 70 Hz, and during a period of approximately five seconds, the high/low frequency alternates once.

The rectifier, wave filter, and voltage stabilizer circuit 5 is a circuit that consists of two diodes D2, a rectifier D3, a voltage stabilizing integrated circuit U3, resistors R24 and R25, and capacitors C6 and C7.

The LED output indication circuit 6 consists of a transistor Q4, resistors R3 and R5, and a light emitting diode LED1, and is utilized to commence time indication.

The voltage output circuit 7 consists of a transistor Q6, a variable resistor VR1, a resistor R15, a transformer T1, and a jumper switch J3, and is utilized to regulate the amount of outputted voltage and increase output efficiency such that after pulse wave output through the transformer T1, a positive/negative pulse wave is generated.

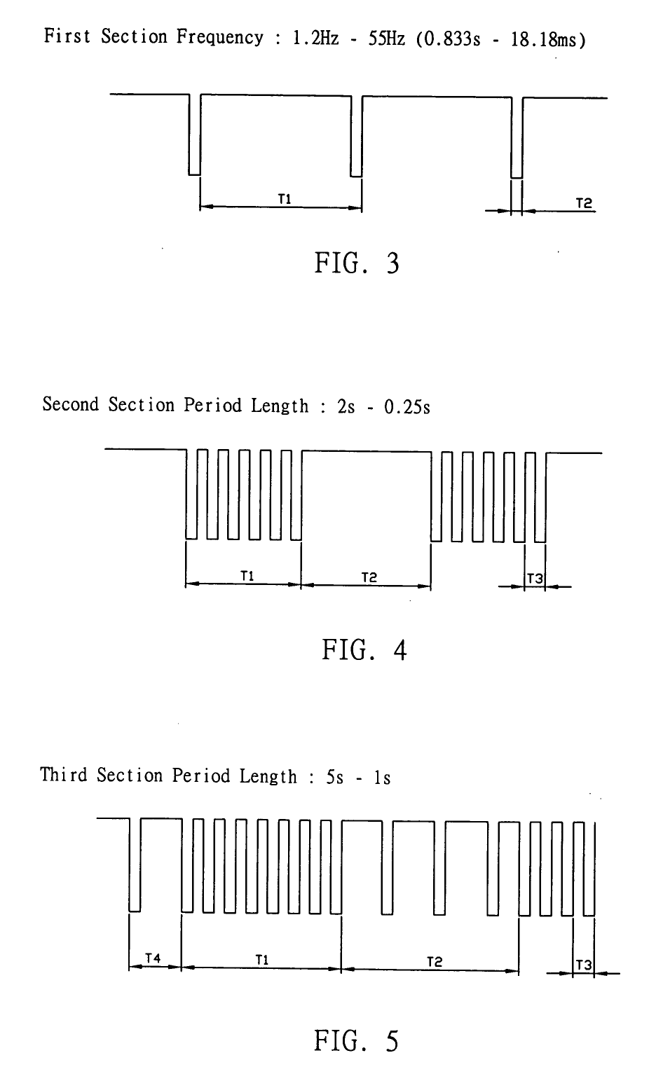

As for the operating method when the said electrical frequency mechanism circuit is switched on, FIG. 3 is the drawing of the first section pure pulse wave, FIG. 4 is the drawing of the second section intermittent wave, and FIG. 5 is the third section high/low frequency alternating wave; first, after voltage is supplied to the input terminal, a transistor Q3 and a resistor R2 via a variable resistor VR2 alters the voltage and adjusts for three types of output wave form frequencies, changing the internal resistance of the transistor Q3, which is utilized to adjust the oscillation frequency of the integrated circuit U1; J1 is capable of changing pulse wave width via resistance and the selector switch, square waves are then generated after control by the square wave oscillator circuit, the switch circuit, and the frequency circuit, resulting in three types of adjustable and switchable pulse wave forms, the pulse switch selector 1 (SW1) capable of toggling between the three types of pulse wave forms, and if the first section fundamental pulse wave is generated by the IC oscillator, resistance is varied to change transistor and resistance until the oscillation frequency of the oscillator is adjusted, thereby producing the fundamental pulse wave; the second section intermittent wave occurs through the interconnection of the second section oscillator circuit and the IC (U1) to generate high-frequency wave forms; the third section high/low frequency alternating wave occurs through the interconnection of the third section oscillator circuit and the IC (U1) to generate high-frequency and low-frequency alternating waves; the said three types of pulse wave wave forms can be measured at the output terminal circuit J2 position and, furthermore, the output terminals are externally connected to electrodes utilizing low magnetic flux by first charging magnetically and afterwards electrical discharge principles, the pulse wave thereby triggering the generation of different positive and negative waves and, as the magnitude of the triggering current and voltage are varied, the resistance under load of the upper half wave and the lower half wave are altered.

In summation of the foregoing section, since the said structural arrangement of the invention herein is demonstrably workable based on the embodiment, is simple and safe in terms of utilization, affords enhanced overall electrotherapeutic efficacy and, furthermore, the present invention is already successfully developed, the invention herein is submitted to the patent bureau for review and the granting of the commensurate patent rights.

1. A physical electrotherapy device comprised of:
A pulse switch selector utilized to selectively toggle between three different outputted wave forms:
A first section pure pulse wave consisting of an integrated circuit U1, U1 internally including reverse logic gates U2C and U2D, resistors R13 and R16, and a capacitor C3 that enable the generation of a fundamental pulse wave, the lowest frequency of which is 1.2 Hz; this section toggled by the said pulse switch selector.
A second section that is an intermittent pulse wave generating oscillator 3 consisting of a square wave oscillator circuit, an intermittent wave outputting fundamental frequency circuit, and a pulse wave frequency control circuit, wherein the square wave oscillator circuit structure consists of reverse logic gates U2E and U2F, a capacitor C5, and resistors R21 and R22; when the second section intermittent wave has output, the fundamental wave frequency consists of control by a transistor Q5 and resistors R4, R7, and R14; the pulse wave frequency circuit structure consists of a controlling transistor Q8 and resistors R20 and R22, and the switch circuit structure consists of a controlling transistor Q7 and a resistor R18, which are utilized to control the second section intermittent wave form, this section utilizing the pulse switch selector (SW1) for toggling, the fundamental frequency of which is approximately 70 Hz and the slowest period is one alternation every two seconds.
A third section that is a high/low frequency alternating wave generating oscillator 4 consisting of a square wave oscillator circuit, a high/low frequency control circuit, and a pulse wave frequency control circuit, wherein the square wave oscillator circuit structure consists of reverse logic gates U2A and U2B, a capacitor C8, and resistors R26 and R28; the high/low frequency control circuit consists of transistors Q1 and Q2 and resistors R8, R9, R11, and R12 that shape the third section high/low frequency alternating waves, and the pulse wave frequency circuit consists of a controlling transistor Q9 and resistor R27, this section utilizing the pulse switch selector (SW1) for toggling, the slowest duration, low frequency of which is approximately 1 Hz, the highest frequency is approximately 70 Hz, and during a period of approximately five seconds, the high/low frequency alternates once.
A rectifier, wave filter, and voltage stabilizer circuit 5, a circuit that consists of two diodes D2, a rectifier D3, a voltage stabilizing integrated circuit U3, resistors R24 and R25, and capacitors C6 and C7.
An LED output indication circuit 6 consisting of a transistor Q4, resistors R3 and R5, and a light emitting diode LED1, and is utilized to commence time indication.
A voltage output circuit 7 consisting of a transistor Q6, a variable resistor VR1, a resistor RI 5, a transformer T1, and a jumper switch J3, and is utilized to regulate the amount of outputted voltage and increase output efficiency such that after pulse wave output through the transformer T1, a positive/negative pulse wave is generated.
As for the operating method when the said electrical frequency mechanism circuit is switched on, voltage is supplied to the input terminal, a variable resistor VR2 alters the voltage and adjusts for three types of output wave form frequencies, square waves are then executed after control by the square wave oscillator circuit, the switch circuit, and the frequency circuit; the said pulse switch selector (SW1) is capable of toggling between the three types of pulse wave forms, the said three types of pulse wave wave forms can be measured at the output terminal circuit J2 position and, furthermore, the output terminals are externally connected to electrodes and after first being magnetically charged, completion is by electrical discharge principles, and following pulse wave triggering, different positive and negative waves are generated and as the magnitude of the triggering current and voltage are varied, the resistance under load of the upper half wave and the lower half wave is altered, thereby automatically adjusting the upper and lower wave width as well as the outputted voltage and current.
